(19) United States
(12) Patent Application Publication
(10) Pub. No.: US 2025/0371703 A1
(43) Pub. Date: Dec. 4, 2025

(54) ELECTRONIC DEVICE FOR ANALYZING SKIN BASED ON HYPERSPECTRAL IMAGE SENSOR AND OPERATING METHOD THEREOF

(71) Applicants: SAMSUNG ELECTRONICS CO., LTD., Suwon-si (KR); CHUNG ANG University Industry Academic Cooperation Foundation, Seoul (KR)

(72) Inventors: Suyeon LEE, Suwon-si (KR); Hojung KIM, Suwon-si (KR); Unjeong KIM, Suwon-si (KR); Hyungbin SON, Seoul (KR)

(73) Assignees: SAMSUNG ELECTRONICS CO., LTD., Suwon-si (KR); CHUNG ANG University Industry Academic Cooperation Foundation, Seoul (KR)

(21) Appl. No.: 19/025,719

(22) Filed: Jan. 16, 2025

(30) Foreign Application Priority Data
May 31, 2024 (KR) 10-2024-0071787

(51) Int. Cl. G06T 7/00; A61B 5/00

(52) U.S. Cl. CPC G06T 7/0012; A61B 5/0075; A61B 5/441; G06T 2207/30088

(57) ABSTRACT

Provided is an electronic device configured to, based on a hyperspectral image sensor, analyze skin, the electronic device including at least one light source configured to emit light at a plurality of angles of incidence with respect to a skin region being measured, a hyperspectral image sensor configured to acquire a plurality of reflection signals corresponding to the light that is emitted at the plurality of angles of incidence and is reflected from the skin region being measured, and at least one processor electrically connected to the at least one light source and the hyperspectral image sensor, and configured to generate a plurality of hyperspectral images based on the plurality of reflection signals acquired through the hyperspectral image sensor, measure reflectance changes based on the plurality of angles of incidence, from the generated plurality of hyperspectral images, and generate skin analysis data, based on the measured reflectance changes.

### CROSS-REFERENCE TO RELATED APPLICATION

This application claims priority to Korean Patent Application No. 10-2024-0071787, filed on May 31, 2024, in the Korean Intellectual Property Office, the disclosure of which is incorporated by reference herein in its entirety.

### BACKGROUND

### 1. Field

Embodiments of the present disclosure relate to an electronic device that acquires and analyzes skin-related data based on a hyperspectral image sensor and an operating method thereof.

### 2. Description of Related Art

Various methods have been proposed to non-invasively measure skin condition. In particular, recently, as user demand relating to beauty has increased, the development of skin diagnosis devices has increased. Because skin condition is an important marker of a user's overall health and well-being, continuous preventive monitoring is necessary to maintain good skin health.

However, the result data of conventional skin condition measurement devices may be affected by the measurement environment, so the reliability and accuracy of the result data may be partially reduced.

### SUMMARY

One or more embodiments provide an electronic device for acquiring skin-related data capable of quantitative analysis using a light source that emits light to have a plurality of angles of incidence and a hyperspectral image sensor and an operating method of the same.

The technical problems to be achieved are not limited to the above technical problems, and other technical problems may be inferred from the following embodiments.

Additional aspects will be set forth in part in the description which follows and, in part, will be apparent from the description, or may be learned by practice of the one or more embodiments.

According to an aspect of one or more embodiments, there is provided an electronic device configured to, based on a hyperspectral image sensor, analyze skin, the electronic device including at least one light source configured to emit light at a plurality of angles of incidence with respect to a skin region being measured, a hyperspectral image sensor configured to acquire a plurality of reflection signals corresponding to the light that is emitted at the plurality of angles of incidence from the at least one light source and is reflected from the skin region being measured, and at least one processor electrically connected to the at least one light source and the hyperspectral image sensor, wherein the at least one processor is configured to generate a plurality of hyperspectral images based on the plurality of reflection signals acquired through the hyperspectral image sensor, measure reflectance changes based on the plurality of angles of incidence, from the generated plurality of hyperspectral images, and generate skin analysis data, based on the measured reflectance changes.

The at least one processor may be further configured to generate, based on the measured reflectance changes, the skin analysis data including at least one of thickness of skin tissue, melanin concentration in skin tissue, moisture content, blood volume, hemoglobin concentration in blood, tumor size, and tumor location.

The at least one light source may include a plurality of light sources respectively configured to emit the light at the plurality of angles of incidence with respect to the skin region being measured.

The at least one light source may include one light source configured to rotate to emit the light at the plurality of angles of incidence with respect to the skin region being measured.

The hyperspectral image sensor may be further configured to acquire the plurality of reflection signals based on a snapshot method of dividing into a plurality of spectral regions and simultaneously measuring the plurality of spectral regions.

The at least one processor, through the hyperspectral image sensor, may be further configured to acquire a first plurality of reflection signals of light that is emitted from the at least one first light source configured to emit light at a first angle of incidence and is reflected from the skin region being measured, and acquire, after the first plurality of reflection signals are acquired, a second plurality of reflection signals of light that is emitted from at least one second light source configured to emit light at a second angle of incidence different from the first angle of incidence and is reflected from the skin region being measured.

The hyperspectral image sensor may be further configured to acquire the plurality of reflection signals based on a line scanning method in which measuring is performed through a single line sensor while moving along a y-axis.

The at least one processor, by the hyperspectral image sensor at a first position, is further configured to acquire a plurality of reflection signals of light that is emitted from at least one first light source configured to emit light at a first angle of incidence and at least one second light source configured to emit light at a second angle of incidence different from the first angle of incidence and is reflected from the skin region being measured, and acquire, based on the hyperspectral image sensor being at a second position different from the first position, a plurality of reflection signals of light that is emitted from the at least one first light source and the at least one second light source and is reflected from the skin region being measured.

The at least one processor, by the hyperspectral image sensor arranged to be movable from a first position to a second position, is further configured to acquire a plurality of reflection signals of light that is emitted from the at least one first light source configured to emit light at a first angle of incidence and is reflected from the skin region being measured, by moving the hyperspectral image sensor from the first position to the second position, and acquire a plurality of reflection signals of light that is emitted from the at least one second light source configured to emit light at a second angle of incidence different from the first angle of incidence and is reflected from the skin region being measured, by moving the hyperspectral image sensor from the first position to the second position.

A wavelength band of the at least one light source and the hyperspectral image sensor may be in a range of 450 nm to 2500 nm.

A wavelength band of the at least one light source and the hyperspectral image sensor may be in a range of 450 nm to 1100 nm.

The electronic device may further include a display, wherein the at least one processor may be further configured to output the generated skin analysis data through the display.

According to another aspect of one or more embodiments, there is provided an operating method of an electronic device configured to, based on a hyperspectral image sensor, analyze skin, the operating method including emitting, by at least one light source, light having a plurality of angles of incidence with respect to a skin region being measured, acquiring, by a hyperspectral image sensor, a plurality of reflection signals of light that is emitted from the at least one light source and is reflected from the skin region being measured, generating a plurality of hyperspectral images based on the acquired plurality of reflection signals, measuring reflectance changes based on the plurality of angles of incidence from the generated plurality of hyperspectral images, and generating skin analysis data based on the measured reflectance changes.

The generating of the skin analysis data may further include, generating, based on the measured reflectance changes, the skin analysis data including at least one of thickness of skin tissue, melanin concentration in skin tissue, moisture content, blood volume, hemoglobin concentration in blood, tumor size, and tumor location.

The hyperspectral image sensor may be further configured to acquire the plurality of reflection signals based on a snapshot method of dividing into a plurality of spectral regions and simultaneously measuring the plurality of spectral regions.

The method may further include acquiring, by the hyperspectral image sensor, a first plurality of reflection signals of light that is emitted from the at least one first light source configured to emit light at a first angle of incidence and is reflected from the skin region being measured, and acquiring, after the first plurality of reflection signals are acquired, a second plurality of reflection signals of light that is emitted from at least one second light source configured to emit light at a second angle of incidence different from the first angle of incidence and is reflected from the skin region being measured.

The hyperspectral image sensor may be further configured to acquire the plurality of reflection signals based on a line scanning method in which measuring is performed by a single line sensor while moving along a y-axis.

The method may further include acquiring, by the hyperspectral image sensor disposed at a first position, a plurality of reflection signals of light that is emitted from at least one first light source configured to emit light at a first angle of incidence and at least one second light source configured to emit light at a second angle of incidence different from the first angle of incidence and is reflected from the skin region being measured, and acquiring, by the hyperspectral image sensor at a second position different from the first position, a plurality of reflection signals of light that is emitted from the at least one first light source and the at least one second light source and is reflected from the skin region being measured.

The method may further include acquiring, by the hyperspectral image sensor arranged to be movable from a first position to a second position, a plurality of reflection signals of light that is emitted from the at least one first light source configured to emit light at a first angle of incidence and is reflected from the skin region being measured, by moving the hyperspectral image sensor from the first position to the second position, and acquiring a plurality of reflection signals of light that is emitted from the at least one second light source configured to emit light at a second angle of incidence different from the first angle of incidence and is reflected from the skin region being measured, by moving the hyperspectral image sensor from the first position to the second position.

According to still another aspect of one or more embodiments, there is provided a non-transitory computer-readable recording medium recording a program for executing a method on a computer, the method including emitting, by at least one light source, light having a plurality of angles of incidence with respect to a skin region being measured, acquiring, by a hyperspectral image sensor, a plurality of reflection signals of light that is emitted from the at least one light source and is reflected from the skin region being measured, generating a plurality of hyperspectral images based on the acquired plurality of reflection signals, measuring reflectance changes based on the plurality of angles of incidence from the generated plurality of hyperspectral images, and generating skin analysis data based on the measured reflectance changes.

### BRIEF DESCRIPTION OF DRAWINGS

The above and other aspects, features, and advantages of one or more embodiments will be more apparent from the following description taken in conjunction with the accompanying drawings, in which:

FIG. 1 is a block diagram of an electronic device according to one or more embodiments;

FIG. 2 is a flowchart illustrating the generation of skin analysis data by an electronic device according to one or more embodiments;

FIG. 3 is a cross-sectional view of an electronic device according to one or more embodiments;

FIG. 4 shows another example cross-sectional view of an electronic device according to one or more embodiments;

FIG. 5 illustrates an example in which changes in reflectance according to multiple angles of incidence are measured by an electronic device according to one or more embodiments;

FIG. 6 is a flowchart of the acquisition of a reflection signal by an electronic device, according to one or more embodiments;

FIG. 7 is a flowchart illustrating the acquisition of a reflected signal by an electronic device, according to one or more other embodiments;

FIG. 8 is a graph showing an absorption material and an absorption spectrum in skin, according to one or more embodiments;

FIG. 9 is a block diagram of an electronic device according to one or more other embodiments;

FIG. 10A a diagram showing an example of the electronic device of FIG. 9; and

FIG. 10B a diagram showing an example of outputting skin analysis data through the electronic device of FIG. 9.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list. For example, the expression, “at least one of a, b, and c,” should be understood as including only a, only b, only c, both a and b, both a and c, both b and c, or all of a, b, and c.

Hereinafter, example embodiments will be described in detail with reference to the accompanying drawings. In the drawings, like reference numerals refer to like components, and a size of each component in the drawings may be exaggerated for clarity and convenience of explanation. The embodiments described below are merely illustrative, and various modifications are possible from these embodiments.

In the following descriptions, when an element or layer is referred to as being “on” or “above” another element or layer, the element or layer may be directly on another element or layer or intervening elements or layers. In the following embodiments, the singular forms include the plural forms unless the context clearly indicates otherwise. When a part “comprises” or “includes” an element in the specification, unless otherwise defined, it is not excluding other elements but may further include other elements. The term “above” and similar directional terms may be applied to both singular and plural.

Also, some embodiments are described in the accompanying drawings in relation to functional blocks, units, and/or modules. Those skilled in the art will understand that such blocks, units, and/or modules are physically implemented by logic circuits, individual components, microprocessors, hard-wired circuits, memory elements, wire connections, and other electronic circuitry. The blocks, units, and/or modules may be formed using semiconductor-based manufacturing techniques or other manufacturing techniques. In the case of the blocks, units, and/or modules implemented by a microprocessor or other similar hardware, the blocks, units, and/or modules may perform various functions discussed in the disclosure by being programmed and controlled using software and may be driven by firmware and/or software. Additionally, each block, unit, and/or module may be implemented by dedicated hardware or may be implemented by a combination of dedicated hardware performing some functions and a processor (e.g., one or more programmed microprocessors and associated circuits) performing other functions. Additionally, in some embodiments, a block, unit, and/or module may be physically separated into two or more individual blocks, units, and/or modules that interact without departing from the scope of the disclosure. Additionally, in some embodiments, blocks, units, and/or modules may be physically combined into more complex blocks, units, and/or modules without departing from the scope of the disclosure.

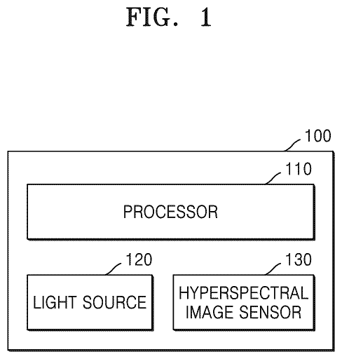

FIG. 1 is a block diagram of an electronic device 100 according to one or more embodiments.

Referring to FIG. 1, the electronic device 100 may include a processor 110, at least one light source 120, and a hyperspectral image sensor 130. However, in the electronic device 100 shown in FIG. 1, only components related to the disclosure are shown. Accordingly, it is obvious to those skilled in the art that the electronic device 100 may further include other components in addition to those shown in FIG. 1.

For example, the electronic device 100 may include a smart phone, a tablet, or a laptop, but is not limited thereto. As another example, the electronic device 100 may be a separate skin analysis device (e.g., a skin diagnosis device).

According to one or more embodiments, the electronic device 100 may include functions to analyze the user's skin. The electronic device 100 may generate skin analysis data based on results obtained through at least one light source 120 and the hyperspectral image sensor 130. For example, the skin analysis data may include at least one of a thickness of skin tissue, a melanin concentration in skin tissue, a moisture content, a blood volume, a hemoglobin concentration in blood, a tumor size, and a tumor location.

According to one or more embodiments, the at least one light source 120 may be arranged to emit light at a plurality of angles of incidence with respect to a skin region being measured of the user. For example, the light source 120 may include a plurality of light sources each arranged to emit light at a plurality of incident angles with respect to the skin region being measured. For another example, the light source 120 may include one light source arranged to be physically rotationally movable to have a plurality of incident angles with respect to the skin region being measured.

According to one or more embodiments, the skin region being measured of the user may be set automatically. For example, the electronic device 100 may automatically set a skin region to be measured in an image (e.g., the user's face area, eye area, cheek area, chin area, forehead area, or neck area, etc.) based on various image processing algorithms.

According to one or more other embodiments, the skin region being measured of the user may be manually set in advance. For example, the electronic device 100 may preset a measurable area through the arrangement of the at least one light source 120 and the hyperspectral image sensor 130.

According to one or more embodiments, the hyperspectral image sensor 130 may acquire a plurality of reflection signals of light that is emitted with a plurality of angles of incidence from the light source 120 and is reflected from the skin region being measured. For example, the hyperspectral image sensor 130 may include a spectrometer that splits a plurality of reflected signals and a converter that converts the split signals into a spectrum.

The hyperspectral image sensors 130 may be classified according to data acquisition type. For example, the hyperspectral image sensor 130 may be classified into a spatial scanning method and a spectral scanning method. The spatial scanning method may include a point-scanning method that simultaneously splits a certain spectral region and scans while moving along spatial coordinates, and a line-scanning method that scans while moving along a y-axis through a single line sensor. In addition, the spectral scanning method includes an area-scanning method that changes a wavelength and scans an entire image of the skin region being measured, and a snapshot method of dividing into a plurality of spectral regions and simultaneously measuring the plurality of spectral regions.

According to one or more embodiments, the processor 110 may generate a plurality of hyperspectral images through a plurality of reflected signals acquired through the hyperspectral image sensor 130. For example, the processor 110 may generate a hyperspectral image in the form of a three-dimensional data cube including spatial information and spectral information through a reflected signal acquired through the hyperspectral image sensor 130. Additionally, the processor 110 may generate a hyperspectral image for each of a plurality of reflected signals in which light having different incident angles is reflected from the skin region being measured.

According to one or more embodiments, the processor 110 may measure the changes in reflectance according to a plurality of angles of incidence from a plurality of generated hyperspectral images. For example, the processor 110 may acquire a first reflection signal hi of light that is emitted from a first light source, has a first incidence angle and is reflected from the skin region being measured, and generates a first hyperspectral image, and a second reflection signal I₂ of light that is emitted from a second light source, has a second incidence angle and is reflected from the skin region being measured and generates a second hyperspectral image. Also, the processor 110 may calculate (obtain) the change in reflectance according to an angle of incidence based on the first hyperspectral image and the second hyperspectral image. The processor 110 may generate skin analysis data (e.g., skin tissue thickness, concentration of absorbed substances in the skin, etc.) through changes in the calculated (obtained) reflectance.

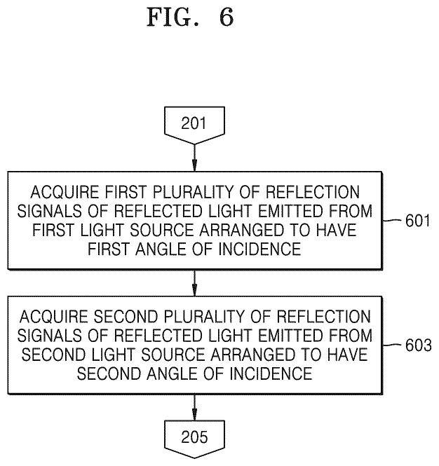

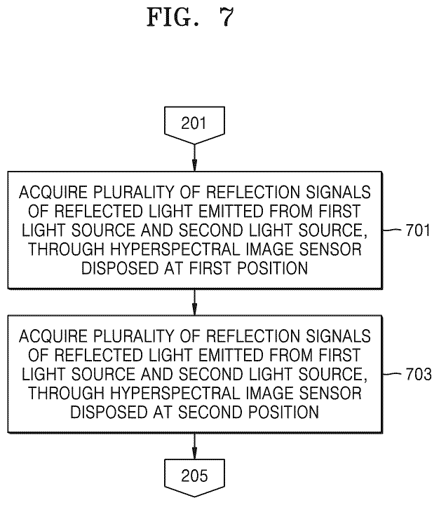

According to one or more embodiments, the processor 110 may perform the operation order of the at least one light source 120 and the hyperspectral image sensor 130 differently depending on the data acquisition type of the hyperspectral image sensor. A detailed description of the operation order will be provided later with reference to FIGS. 6 and 7.

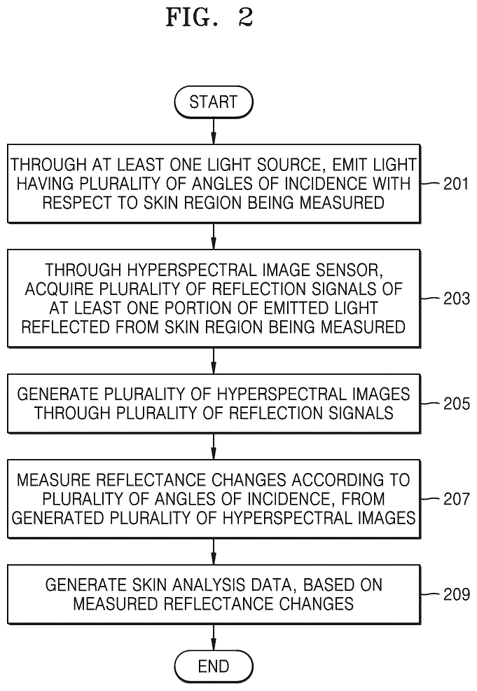

FIG. 2 is a flowchart illustrating the generation of skin analysis data by an electronic device according to one or more embodiments.

Referring to FIG. 2, in operation 201, a processor (e.g., the processor 110 of FIG. 1) of an electronic device (e.g., the electronic device 100 of FIG. 1) may emit light having a plurality of incident angles with respect to a skin region being measured through at least one light source (e.g., the at least one light source 120 of FIG. 1).

According to one or more embodiments, the at least one light source 120 may be arranged to emit light at a plurality of angles of incidence with respect to the skin region being measured of the user. For example, the at least one light source 120 may include a first light source with an incident angle of 0° degree, a second light source with an incident angle of approximately 5°, and a third light source with an incident angle of approximately 10° with respect to the skin region being measured. For another example, the at least one light source 120 may include only a first light source arranged to be physically rotationally movable so that the angle of incidence with respect to the skin region being measured is 0°, about 5°, and about 10°.

According to one or more embodiments, in operation 203, the processor 110 may obtain a plurality of reflection signals of at least one light that is emitted through a hyperspectral image sensor (e.g., hyperspectral image sensor 130 of FIG. 1) and is reflected from the skin region being measured. The plural reflection signals may include reflection spectrum values obtained by reflecting light emitted at different angles of incidence from the skin region being measured.

According to one or more embodiments, the electronic device 100 may filter changes in the reflection spectrum by a measurement environment and detect only changes in the reflection spectrum by a concentration of substances in the skin, the thickness of skin tissue, etc. by acquiring reflected signals of light emitted from a light source with multiple angles of incidence compared to a conventional device that measures through a light source with a single angle of incidence. For example, when a hyperspectral image of a skin is generated using a conventional device that measures through a light source with a single angle of incidence, the distribution and changes of various elements in the skin may be detected from the generated hyperspectral image, but there are limitations to quantitative analysis because an absolute value of the reflection spectrum may be affected by a measurement environment, such as measurement of facial curves, etc. Accordingly, the electronic device 100 according to one or more embodiments may generate a hyperspectral image based on a light source having a plurality of incident angles and filter changes in the reflection spectrum by the measurement environment.

According to one or more embodiments, in operation 205, the processor 110 may generate a plurality of hyperspectral images through a plurality of reflection signals. For example, the processor 110 may generate a hyperspectral image in the form of a three-dimensional data cube including spatial information and spectral information including a continuous spectrum for each pixel through a reflection signal acquired through the hyperspectral image sensor 130. Additionally, the processor 110 may generate a hyperspectral image for each of a plurality of reflection signals of light having different incident angles and reflected from the skin region being measured.

According to one or more embodiments, the processor 110 may store hyperspectral images according to different angles of incidence in a separate memory. The memory may be configured to store one or more instructions. For example, memory 220 may include on-chip memory, cache memory, random access memory (RAM), read only memory (ROM), flash memory, solid state drive (SSD), hard disk drive (HDD), or ODD (Optical Disc Drive), but is not limited thereto.

According to one or more embodiments, the processor 110 may measure the change in reflectance according to a plurality of incident angles from the plurality of hyperspectral images generated in operation 207. According to one or more embodiments, the processor 110 may generate skin analysis data based on the change in reflectance measured in operation 209.

For example, the processor 110 may acquire a first reflection signal I₁ of light that is emitted from a first light source having a first incident angle and is reflected from the skin region being measured and generates a first hyperspectral image, and may acquire a second reflection signal I₂ of light that is emitted from a second light source having a second incident angle and is reflected from the skin region being measured and generate a second hyperspectral image.

Additionally, the processor 110 may calculate (obtain) the change in reflectance according to an angle of incidence based on the first hyperspectral image and the second hyperspectral image. The processor 110 may detect various elements in the skin according to [Equation 1] based on the calculated (obtained) change in reflectance.

$${l}_{1} / {I}_{N} = f ( θ , n , x ) \qquad [ Equation 1 ]$$

In [Equation 1], I₁ refers to a first reflection signal (or first reflection spectrum value) of light that is emitted from a first light source with a first incident angle and is reflected from a skin region being measured, and I_{N} refers to an N^{th} reflection signal (or N^{th} reflection spectrum value) of light that is emitted from an N^{th} light source (N is an integer of 2 or more) and is reflected from the skin region being measured.

Additionally, the function f may be a function of values related to angles of incidence greater than 0°, materials within the skin area, and various elements within the skin. For example, θ refers to an angle of incidence, n refers to a measured value related to a material in the skin region being measured, and x refers to a value related to various elements in the skin (e.g., thickness of skin tissue, concentration of absorbed material in the skin).

For example, if the electronic device 100 includes two light sources, the first angle of incidence of the first light source is 0° and the second angle of incidence of the second light source is 5°, the processor 110 may acquire values related to various elements in the skin by measuring the changes in reflectance of the first reflection signal I₁ and the second reflection signal I₂. As another example, if the electronic device 100 includes three light sources, a first angle of incidence of a first light source is 0°, a second angle of incidence of a second light source is 5°, and a third angle of incidence of a third light source is 10°, the processor 110 may acquire values related to various elements in the skin by measuring the changes in reflectance of the first reflection signal I₁ and the second reflection signal I₂ and the changes in reflectance of the first reflection signal I₁ and the third reflection signal I₃.

According to one or more embodiments, the processor 110 may generate skin analysis data based on values related to various elements in the skin acquired by measuring the changes in reflectance of reflection signals. For example, if the various elements within the skin include a skin tissue thickness (d) and a concentration of absorbed material within the skin (c), the values related to the various elements in the skin may denote a multiplication value of the skin tissue thickness (d) and the concentration (c) of the absorbed material in the skin.

Accordingly, when it is assumed that the thickness of skin tissue of the skin region being measured is the same, the value acquired through the change in reflectance of the reflection signals may be a concentration value of absorbed materials (e.g., hemoglobin, melanin, moisture) in the skin.

Additionally, when it is assumed that the concentration of the absorbed material in the skin of the skin region being measured is the same, a value acquired through the change in reflectance of the reflection signals may be a thickness of the skin tissue.

However, various components in the skin are not limited to the thickness of skin tissue and concentration of absorbed materials in the skin but may include the location and size of a tumor when a tumor within the skin is included.

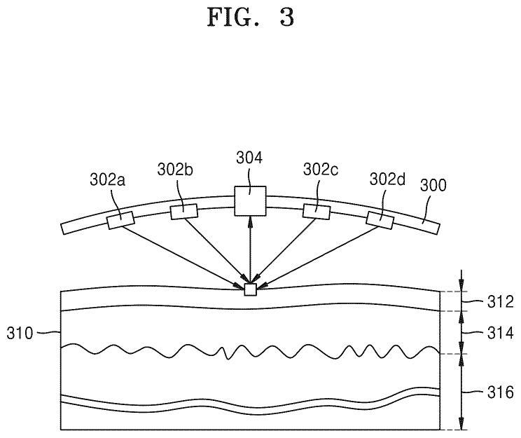

FIG. 3 is a cross-sectional view of an electronic device 300 according to one or more embodiments.

Referring to FIG. 3, the electronic device 300 may include four light sources 302a, 302b, 302c, and 302d and a hyperspectral image sensor 304. For convenience of explanation, the processor (e.g., processor 110 of FIG. 1) is omitted, but components included in the electronic device 300 of FIG. 3 may correspond to the components included in the electronic device 100 of FIG. 1. Also, for convenience of explanation, it is depicted that the electronic device 300 of FIG. 3 includes four light sources, but the number of light sources included in the electronic device 300 is not limited thereto and may include two or more light sources.

According to one or more embodiments, the electronic device 300 may generate user skin analysis data for a skin region being measured 310.

The skin region being measured 310 includes a stratum corneum 312 with a thickness of about 10 μm, an epidermis 314 with a thickness of about 100 μm, and the dermis 316 with a thickness of about 4 mm, and the penetration depth and absorption location of light may be determined by various absorption factors such as, melanin, hemoglobin, and moisture in the skin.

For example, hemoglobin present in blood may be mainly distributed in the dermis 316, and melanin may be mainly distributed in the epidermis 314. In addition, the stratum corneum 312 may transmit wavelength bands in a range from about 150 nm to about 470 nm, the epidermis 314 may transmit wavelength bands in a range from about 470 nm to about 600 nm and in a range from about 940 nm to about 1,100 nm, and the dermis 316 may transmit wavelength bands in a range from about 600 nm to about 940 nm.

According to one or more embodiments, the four light sources 302a, 302b, 302c, and 302d may each be arranged to emit light at a plurality of incident angles with respect to the skin region being measured 310 of the user. For example, the four light sources 302a, 302b, 302c, and 302d may each be arranged to emit light at an incident angle greater than 0° and different from each other. The four light sources 302a, 302b, 302c, and 302d may each emit light in different wavelength bands or may emit single light in multiple wavelength bands.

According to one or more embodiments, the hyperspectral image sensor 304 may acquire four reflection signals of light that is emitted from the four light sources 302a, 302b, 302c, and 302d and is reflected from a specific region (pixel) of the skin region being measured 310. The reflection signal may denote a reflection spectrum value acquired by reflecting light emitted from a light source from the skin region being measured 310.

For example, a processor (e.g., processor 110 of FIG. 1) may generate four hyperspectral images through the four reflection signals acquired through the hyperspectral image sensor 304.

The processor 110 may generate four hyperspectral images of the entire skin region being measured 310 by repeating the operation of acquiring the reflection signal through the four light sources 302a, 302b, 302c, and 302d. Afterwards, the processor 110 may calculate (obtain) the change in reflectance according to an angle of incidence based on the four generated hyperspectral images and acquire values related to various elements in the skin (e.g., thickness of skin tissue, concentration of absorbed substances in the skin).

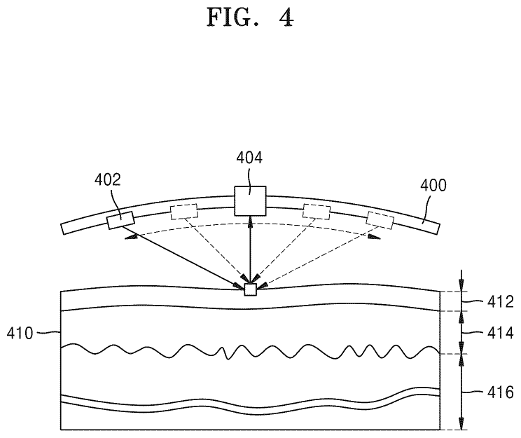

FIG. 4 shows another cross-sectional view of an electronic device 400 according to one or more embodiments.

Referring to FIG. 4, the electronic device 400 may include one light source 402 and a hyperspectral image sensor 404. For convenience of explanation, the processor (e.g., processor 110 of FIG. 1) is omitted, but the components included in the electronic device 400 of FIG. 4 may correspond to components included in the electronic device 100 of FIG. 1. In addition, for convenience of explanation, the electronic device 400 in FIG. 4 is shown as one light source physically rotating to four positions, but the number of positions to which the light source of the electronic device 400 rotates is not limited thereto and may rotate to two or more positions.

According to one or more embodiments, the electronic device 400 may generate user skin analysis data for a skin region being measured 410.

The skin region being measured 410 includes a stratum corneum 412, an epidermis 414, and a dermis 416, and the penetration depth and absorption location of light may be determined by various absorption factors, such as melanin, hemoglobin, and moisture in the skin.

According to one or more embodiments, the one light source 402 may be arranged to be physically rotationally movable to have a plurality of incident angles with respect to the skin region being measured 410 of the user. For example, the one light source 402 may be arranged to physically rotate on one side of the electronic device 400 to have an incident angle greater than 0° and different from each other. The one light source 402 may emit light in multiple wavelength bands.

According to one or more embodiments, the hyperspectral image sensor 404 may acquire a reflection signal of light that is emitted while the light source 402 rotates and moves and is reflected from a specific region (pixel) of the skin region being measured 410. For example, as the light source 402 rotates and emits light at four positions, the processor (e.g., processor 110 in FIG. 1) may generate four hyperspectral images using four reflection signals acquired through the hyperspectral image sensor 404.

The processor 110 may generate four hyperspectral images for the entire skin region being measured 410 by repeating the rotational movement of the light source 402 and the operation of acquiring the reflection signal. Afterwards, the processor 110 may calculate (obtain) the change in reflectance according to an angle of incidence based on the four generated hyperspectral images and acquire values related to various elements in the skin (e.g., thickness of skin tissue, concentration of absorbed substances in the skin).

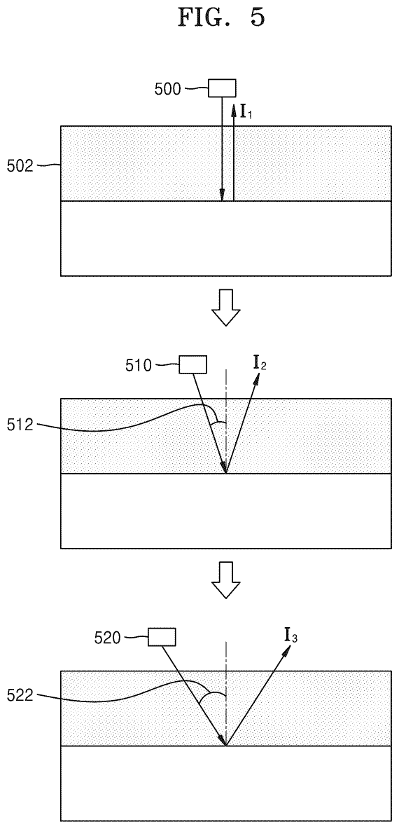

FIG. 5 illustrates an example of measuring reflectance change according to multiple angles of incidence by an electronic device according to one or more embodiments.

Referring to FIG. 5, light sources 500, 510, and 520 of the electronic device (e.g., the electronic device 100 of FIG. 1) may be arranged to emit light at a plurality of incident angles with respect to a skin region being measured 502. For example, the first light source 500 may be arranged to emit light at a first angle of incidence (e.g., 0°) with respect to the skin region being measured 502, the second light source 510 may be arranged to emit light at a second angle of incidence (e.g., about 5°) with respect to the skin region being measured 502, and the third light source 520 may be arranged to emit light at a third angle of incidence 522 (e.g., about 10°) with respect to the skin region being measured 502.

According to one or more embodiments, the light sources 500, 510, and 520 may each be disposed on one side of the electronic device 100 and sequentially emit light. For example, after light is emitted through the first light source 500, light may be emitted in the order of the second light source 510 and the third light source 520. In one or more other embodiments, the light sources 500, 510, and 520 may be a single light source disposed on one side of the electronic device 100 and may emit light while moving their positions through a physical rotational movement. For example, when the light sources 500, 510, and 520 are a single light source, the single light source may emit light while moving from a first position (e.g., a position corresponding to the first light source 500), a second position (e.g., a position corresponding to the second light source 510), and a third position (e.g., a position corresponding to the third light source 520).

According to one or more embodiments, a hyperspectral image sensor (e.g., the hyperspectral image sensor 130 of FIG. 1) may acquire reflection signals 11, 12, and Is generated by reflecting light emitted from the light sources 500, 510, and 520 from the skin region being measured 502. Light emitted from each of the light sources 500, 510, and 520 may be reflected from the skin region being measured 502 and have different reflection spectrum values. Additionally, light emitted from the light sources 500, 510, and 520 may have different reflection spectrum values depending on the thickness of the skin region being measured 502 and the concentration of substances within the area, etc.

For example, light emitted from light sources 500, 510, and 520 may each have different reflection spectrum values even when the light is reflected from the skin region being measured 502 having the same thickness. Accordingly, the processor (e.g., processor 110 in FIG. 1) may acquire a material concentration value in the skin region being measured 502 based on the different reflection spectrum values. As another example, light emitted from each of the light sources 500, 510, and 520 may have different reflection spectrum values even if the light is reflected from the skin region being measured 502, which has the same material concentration value within the region. Accordingly, the processor 110 may acquire a thickness of the skin region being measured 502 based on the different reflection spectrum values.

FIG. 6 is a flowchart of the acquisition of a reflection signal by an electronic device according to one or more embodiments. FIG. 6 is a flowchart explaining operation 203 of FIG. 2.

Referring to FIG. 6, the data acquisition form of a hyperspectral image sensor (e.g., the hyperspectral image sensor 130 of FIG. 1) included in an electronic device (e.g., the electronic device 100 of FIG. 1) is a spectral scanning method. For example, the hyperspectral image sensor 130 may include a snapshot method of dividing into a plurality of spectral regions and simultaneously measuring the plurality of spectral regions.

According to one embodiment, in operation 601, the processor (e.g., the processor 110 of FIG. 1) of the electronic device 100 may acquire a first plurality of reflection signals of light emitted from a first light source arranged to emit light at a first angle of incidence. According to one or more embodiments, the processor 110 may generate a first hyperspectral image based on the acquired first plurality of reflection signals. For example, the processor 110 may acquire the first plurality of reflection signals through the hyperspectral image sensor 130 by controlling light to be emitted from the first light source having a first angle of incidence. The first plurality of reflection signals may have different spectral values depending on the skin thickness of the user's skin region being measured and the concentration of substances in the skin.

According to one or more embodiments, in operation 603, the processor 110 may acquire a second plurality of reflection signals of reflected light emitted from a second light source arranged to emit light at a second incident angle. According to one or more embodiments, the processor 110 may generate a second hyperspectral image based on the acquired second plurality of reflection signals. For example, the processor 110 may acquire a second plurality of reflection signals through the hyperspectral image sensor 130 by controlling light to be emitted from a second light source having a second incident angle that is different from the first incident angle. The second plurality of reflection signals may have different spectral values depending on the thickness of the skin region being measured of the user and the concentration of substances in the skin.

According to one or more embodiments, the processor 110 may sequentially perform operations 601 and 603. The processor 110 may generate a first hyperspectral image through the first light source having a first incident angle and the hyperspectral image sensor 130, and afterwards, may generate a second hyperspectral image through a second light source having a second incident angle and the hyperspectral image sensor 130.

FIG. 7 is a flowchart of the acquisition of a reflection signal by an electronic device according to one or more embodiments. FIG. 7 is a flowchart specifically explaining operation 203 of FIG. 2.

Referring to FIG. 7, the data acquisition form of a hyperspectral image sensor (e.g., hyperspectral image sensor 130 of FIG. 1) included in an electronic device (e.g., electronic device 100 of FIG. 1) is a spatial scanning method. For example, the hyperspectral image sensor 130 may include a line scanning method in which a single line sensor moves along a y-axis and measures spatial regions.

According to one or more embodiments, in operation 701, a processor (e.g., the processor 110 of FIG. 1) of the electronic device 100 may acquire a plurality of reflection signals reflected light emitted from a first light source and a second light source through the hyperspectral image sensor 130 disposed at a first position. The first light source and the second light source may emit light having different angles of incidence. According to one or more embodiments, the processor 110 may generate a first hyperspectral image based on the plurality of acquired reflection signals. For example, the processor 110 may acquire a plurality of reflection signals through the hyperspectral image sensor 130 disposed at the first position by controlling light to be sequentially emitted from the first light source having a first incident angle and the second light source having a second incident angle.

According to one or more embodiments, in operation 703, the processor 110 may acquire a plurality of reflection signals of reflected light emitted from the first light source and the second light source through the hyperspectral image sensor 130 disposed at the second position. According to one or more embodiments, the processor 110 may generate a second hyperspectral image based on the acquired plurality of reflection signals. For example, the processor 110 may acquire a plurality of reflection signals through the hyperspectral image sensor 130 disposed at the second position by controlling light to be sequentially emitted from the first light source having a first incident angle and the second light source having a second incident angle.

According to one or more other embodiments, the processor 110 of the electronic device 100 may acquire a plurality of reflection signals of light that is emitted from at least one first light source arranged to emit light at a first angle of incidence and is reflected from the skin region being measured through the hyperspectral image sensor 130 arranged to be movable from the first position to the second position. Additionally, the processor 110 may acquire a plurality of reflection signals of light that is emitted from at least one second light source arranged to emit light at a second incident angle different from the first incident angle and is reflected from the skin region being measured. For example, each of the plurality of reflection signals may be acquired by moving the hyperspectral image sensor 130 from the first position to the second position.

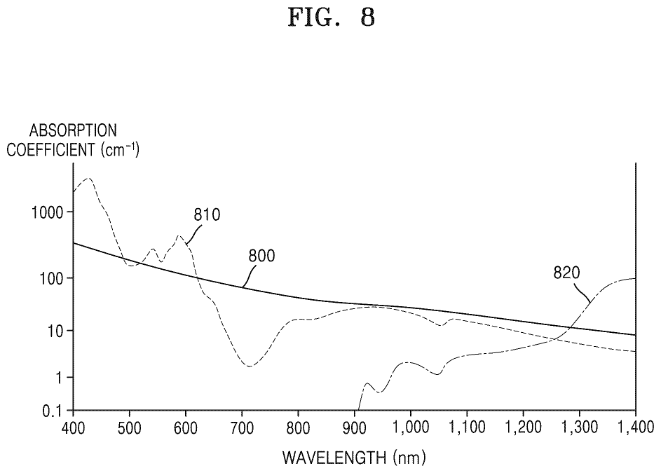

FIG. 8 is a graph showing an absorption material and an absorption spectrum in the skin, according to one or more embodiments.

Referring to FIG. 8, the absorption coefficient in a wavelength band may be different for each absorbent material in the skin.

For example, melanin 800 and hemoglobin 810 in the skin may absorb wavelengths in a range of about 400 nm to about 1400 nm, and in particular, hemoglobin 810 may have a high absorption spectrum in a lower wavelength range (e.g., from about 400 nm to about 700 nm).

Also, moisture 820 in the skin may absorb wavelengths in a range from about 900 nm to about 1400 nm and does not absorb wavelengths in a lower range (e.g., from about 400 nm to about 900 nm).

Accordingly, even when light emitted from at least one light source (e.g., the light source 120 of FIG. 1) is reflected from a skin region being measured, the processor (e.g., processor 110 in FIG. 1) may acquire different reflection spectrum values depending on the concentration value of substances in the skin region being measured. Additionally, a wavelength band of the light source 120 and a hyperspectral image sensor (e.g., the hyperspectral image sensor 130 of FIG. 1) may be in a range from about 450 nm to about 2500 nm, and preferably may be in a range from about 450 nm to about 1100 nm.

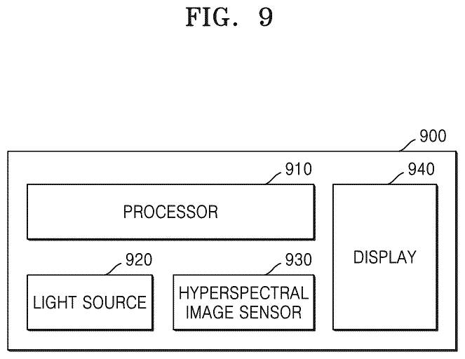

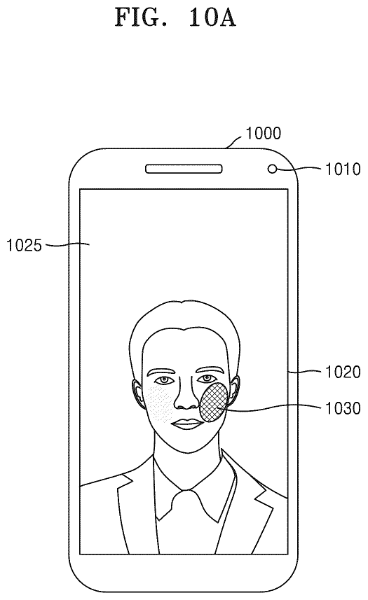

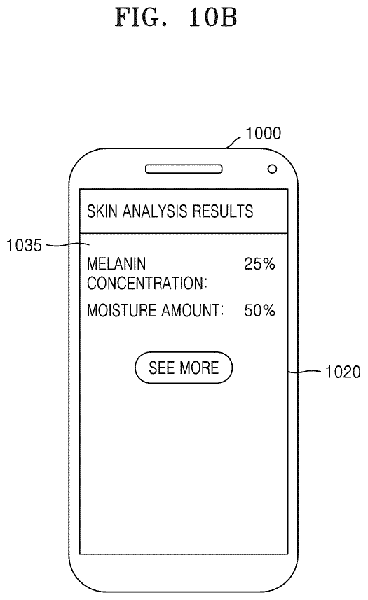

FIG. 9 is a block diagram of an electronic device 900 according to one or more other embodiments. FIG. 10A is a diagram showing an example of the electronic device 900 of FIG. 9. FIG. 10B is a diagram showing an example of outputting skin analysis data through the electronic device 900 of FIG. 9.

Referring to FIG. 9, the electronic device 900 may include a processor 910, at least one light source 920, a hyperspectral image sensor 930, and a display 940. The processor 910, the at least one light source 920, and the hyperspectral image sensor 930 may correspond to the processor 110, the at least one light source 120, and the hyperspectral image sensor 130 of FIG. 1, and thus, overlapping descriptions may be omitted.

According to one or more embodiments, the display 940 may visually provide information generated by the electronic device 900 to the user. For example, the display 940 may output the user's skin analysis data to the outside. The display 940 may include a liquid crystal display panel (LCD), an organic light emitting display panel (OLED), or the like.

Referring to FIG. 10A, an electronic device 1000 may include a camera 1010 and a display panel 1020 that outputs user interface (UI) data. For example, a first UI 1025 including the user's image captured through the camera 1010 may be output on the display panel 1020. The first UI 1025 may include a user's skin region being measured 1030 for generating the user's skin analysis data.

Referring to FIG. 10B, the electronic device 1000 may output a second UI 1035 including skin analysis data through the display panel 1020. The skin analysis data may be acquired through the at least one light source 920 and the hyperspectral image sensor 930. For example, when the at least one light source 920 emits light having a plurality of incident angles to the user's skin region being measured 1030 and when the hyperspectral image sensor 930 acquires a plurality of reflection signals reflected from the skin region being measured 1030, the processor 910 may generate skin analysis data by generating a hyperspectral image through the acquired plurality of reflection signals and measuring reflectance changes according to a plurality of angles of incidence. For example, the skin analysis data may display melanin concentration, moisture content, etc. in numerical values, but is not limited thereto.

The embodiments may also be implemented in the form of a recording medium including instructions executable by a computer, such as program modules executed by a computer. Computer-readable media may be any available media that may be accessed by a computer and includes both volatile and non-volatile media, removable and non-removable media. Also, computer-readable media may include computer storage media and communication media. The computer storage media includes both volatile and non-volatile, removable and non-removable media implemented in any method or technology for storage of information such as computer-readable instructions, data structures, program modules, or other data. The communication media typically may include computer-readable instructions, data structures, or other data, such as modulated data signals, or program modules.

Also, a computer-readable storage media may be provided in the form of non-transitory storage media. Here, non-transitory storage medium indicates that it is a tangible device and does not include signals (e.g. electromagnetic waves), and the term does not distinguish between a case when data is stored semi-permanently in a storage medium and a case when data is stored temporarily. For example, a non-transitory storage medium may include a buffer where data is temporarily stored.

According to one or more embodiments, methods according to various embodiments disclosed in the document may be provided and included in a computer program product. Computer program products are commodities and may be traded between sellers and buyers. A computer program product may be distributed in the form of a machine-readable storage medium (e.g. compact disc read only memory (CD-ROM)) or distributed (e.g. downloaded or uploaded) online, through an application store or directly between two user devices (e.g. smartphones). In the case of online distribution, at least a portion of the computer program product (e.g., a downloadable app) may be at least temporarily stored or temporarily created in a device-readable storage medium, such as the memory of a manufacturer's server, an application store server, or a relay server.

The foregoing description of the disclosure is for illustrative purposes, and those skilled in the art to which the disclosure pertains will understand that the disclosure may be easily modified into another specific form without departing its technical idea or essential features. Therefore, the embodiments described above should be understood in all respects as illustrative and not restrictive. For example, each component described as single may be implemented in a distributed manner, and similarly, components described as distributed may also be implemented in a combined form.

The scope of the disclosure is indicated by the claims described below and their equivalents rather than the detailed description above, and all changes or modified forms derived from the meaning and scope of the claims and their equivalent concepts should be construed as being included in the scope of the disclosure.

It should be understood that embodiments described herein should be considered in a descriptive sense only and not for purposes of limitation. Descriptions of features or aspects within each embodiment should typically be considered as available for other similar features or aspects one or more other embodiments. While embodiments have been described with reference to the figures, it will be understood by those of ordinary skill in the art that various changes in form and details may be made therein without departing from the spirit and scope as defined by the following claims and their equivalents.

What is claimed is:

1. An electronic device configured to, based on a hyperspectral image sensor, analyze skin, the electronic device comprising:
at least one light source configured to emit light at a plurality of angles of incidence with respect to a skin region being measured;
a hyperspectral image sensor configured to acquire a plurality of reflection signals corresponding to the light that is emitted at the plurality of angles of incidence from the at least one light source and is reflected from the skin region being measured; and
at least one processor electrically connected to the at least one light source and the hyperspectral image sensor,
wherein the at least one processor is configured to:
generate a plurality of hyperspectral images based on the plurality of reflection signals acquired through the hyperspectral image sensor;
measure reflectance changes based on the plurality of angles of incidence, from the generated plurality of hyperspectral images; and
generate skin analysis data, based on the measured reflectance changes.

2. The electronic device of claim 1, wherein the at least one processor is further configured to generate, based on the measured reflectance changes, the skin analysis data comprising at least one of thickness of skin tissue, melanin concentration in skin tissue, moisture content, blood volume, hemoglobin concentration in blood, tumor size, and tumor location.

3. The electronic device of claim 1, wherein the at least one light source comprising a plurality of light sources respectively configured to emit the light at the plurality of angles of incidence with respect to the skin region being measured.

4. The electronic device of claim 1, wherein the at least one light source comprises one light source configured to rotate to emit the light at the plurality of angles of incidence with respect to the skin region being measured.

5. The electronic device of claim 1, wherein the hyperspectral image sensor is further configured to acquire the plurality of reflection signals based on a snapshot method of dividing into a plurality of spectral regions and simultaneously measuring the plurality of spectral regions.

6. The electronic device of claim 5, wherein the at least one processor, through the hyperspectral image sensor, is further configured to:
acquire a first plurality of reflection signals of light that is emitted from the at least one first light source configured to emit light at a first angle of incidence and is reflected from the skin region being measured; and
acquire, after the first plurality of reflection signals are acquired, a second plurality of reflection signals of light that is emitted from at least one second light source configured to emit light at a second angle of incidence different from the first angle of incidence and is reflected from the skin region being measured.

7. The electronic device of claim 1, wherein the hyperspectral image sensor is further configured to acquire the plurality of reflection signals based on a line scanning method in which measuring is performed through a single line sensor while moving along a y-axis.

8. The electronic device of claim 7, wherein the at least one processor, by the hyperspectral image sensor at a first position, is further configured to:
acquire a plurality of reflection signals of light that is emitted from at least one first light source configured to emit light at a first angle of incidence and at least one second light source configured to emit light at a second angle of incidence different from the first angle of incidence and is reflected from the skin region being measured; and
acquire, based on the hyperspectral image sensor being at a second position different from the first position, a plurality of reflection signals of light that is emitted from the at least one first light source and the at least one second light source and is reflected from the skin region being measured.

9. The electronic device of claim 7, wherein the at least one processor, through the hyperspectral image sensor arranged to be movable from a first position to a second position, is further configured to:
acquire a plurality of reflection signals of light that is emitted from the at least one first light source configured to emit light at a first angle of incidence and is reflected from the skin region being measured, by moving the hyperspectral image sensor from the first position to the second position; and
acquire a plurality of reflection signals of light that is emitted from the at least one second light source configured to emit light at a second angle of incidence different from the first angle of incidence and is reflected from the skin region being measured, by moving the hyperspectral image sensor from the first position to the second position.

10. The electronic device of claim 1, wherein a wavelength band of the at least one light source and the hyperspectral image sensor is in a range of 450 nm to 2500 nm.

11. The electronic device of claim 1, wherein a wavelength band of the at least one light source and the hyperspectral image sensor is in a range of 450 nm to 1100 nm.

12. The electronic device of claim 1, further comprising a display,
wherein the at least one processor is further configured to output the generated skin analysis data through the display.

13. An operating method of an electronic device configured to, based on a hyperspectral image sensor, analyze skin, the operating method comprising:
emitting, by at least one light source, light having a plurality of angles of incidence with respect to a skin region being measured;
acquiring, by a hyperspectral image sensor, a plurality of reflection signals of light that is emitted from the at least one light source and is reflected from the skin region being measured;
generating a plurality of hyperspectral images based on the acquired plurality of reflection signals;
measuring reflectance changes based on the plurality of angles of incidence from the generated plurality of hyperspectral images; and
generating skin analysis data based on the measured reflectance changes.

14. The method of claim 13, wherein the generating of the skin analysis data further comprises:
generating, based on the measured reflectance changes, the skin analysis data comprising at least one of thickness of skin tissue, melanin concentration in skin tissue, moisture content, blood volume, hemoglobin concentration in blood, tumor size, and tumor location.

15. The method of claim 13, wherein the hyperspectral image sensor is further configured to acquire the plurality of reflection signals based on a snapshot method of dividing into a plurality of spectral regions and simultaneously measuring the plurality of spectral regions.

16. The method of claim 15, further comprising:
acquiring, by the hyperspectral image sensor, a first plurality of reflection signals of light that is emitted from the at least one first light source configured to emit light at a first angle of incidence and is reflected from the skin region being measured; and
acquiring, after the first plurality of reflection signals are acquired, a second plurality of reflection signals of light that is emitted from at least one second light source configured to emit light at a second angle of incidence different from the first angle of incidence and is reflected from the skin region being measured.

17. The method of claim 13, wherein the hyperspectral image sensor is further configured to acquire the plurality of reflection signals based on a line scanning method in which measuring is performed by a single line sensor while moving along a y-axis.

18. The method of claim 17, further comprising:
acquiring, by the hyperspectral image sensor disposed at a first position, a plurality of reflection signals of light that is emitted from at least one first light source configured to emit light at a first angle of incidence and at least one second light source configured to emit light at a second angle of incidence different from the first angle of incidence and is reflected from the skin region being measured; and
acquiring, by the hyperspectral image sensor at a second position different from the first position, a plurality of reflection signals of light that is emitted from the at least one first light source and the at least one second light source and is reflected from the skin region being measured.

19. The method of claim 17, further comprising:
acquiring, by the hyperspectral image sensor arranged to be movable from a first position to a second position, a plurality of reflection signals of light that is emitted from the at least one first light source configured to emit light at a first angle of incidence and is reflected from the skin region being measured, by moving the hyperspectral image sensor from the first position to the second position; and
acquiring a plurality of reflection signals of light that is emitted from the at least one second light source configured to emit light at a second angle of incidence different from the first angle of incidence and is reflected from the skin region being measured, by moving the hyperspectral image sensor from the first position to the second position.

20. A non-transitory computer-readable recording medium recording a program for executing a method on a computer, the method comprising:
emitting, by at least one light source, light having a plurality of angles of incidence with respect to a skin region being measured;
acquiring, by a hyperspectral image sensor, a plurality of reflection signals of light that is emitted from the at least one light source and is reflected from the skin region being measured;
generating a plurality of hyperspectral images based on the acquired plurality of reflection signals;
measuring reflectance changes based on the plurality of angles of incidence from the generated plurality of hyperspectral images; and
generating skin analysis data based on the measured reflectance changes.
